(19) United States
(12) Patent Application Publication
(10) Pub. No.: US 2010/0198266 A1
(43) Pub. Date: Aug. 5, 2010

(54) Biplanar Fracture Fixation

(76) Inventor: Paul Fuad Nassab, Parkville, MO (US)

Correspondence Address: Patent Docket Department;Armstrong Teasdale LLP, One Metropolitan Square, Suite 2600, St. Louis, MO, 63102-2740, US

(21) Appl. No.: 12/701,307

(22) Filed: Feb. 5, 2010

Related U.S. Application Data
(60) Provisional application No. 61/150,284, filed on Feb. 5, 2009.

(51) Int. Cl. A61B 17/80

(52) U.S. Cl. 606/280

(57) ABSTRACT

An osteosynthesis device is provided. The device includes a first portion and a second portion coupled to the first portion. The first portion is positioned substantially along a first plane and is configurable to fit a first bone surface. The second portion is positioned substantially along a second plane that is different from the first plane and is configurable to fit a second bone surface.

### CROSS-REFERENCE TO RELATED APPLICATIONS

This application claims the benefit of U.S. Provisional Application No. 61/150,284, filed Feb. 5, 2009, which is hereby incorporated by reference in its entirety.

### BACKGROUND OF THE INVENTION

The subject matter described herein relates generally to osteosynthesis devices and, more particularly, to a biplanar fracture fixation for a fractured bone.

Osteosynthesis devices are known to stabilize and/or couple a fractured bone. Known osteosynthesis devices include a portion, a rod, a screw, a pin, and/or a wire. At least some known osteosynthesis devices are available in a variety of shapes, including flat and contoured. Notably, known osteosynthesis devices are planar and configured to be coupled to a single face of a bone. To provide sufficient support, at least some known osteosynthesis devices include a plurality of openings that are aligned on one surface of a bone to facilitate inserting a plurality of screws and/or pins through the plurality of openings and couple the osteosynthesis device to the bone.

### BRIEF SUMMARY OF THE INVENTION

In one aspect, a method for osteosynthesis is provided. The method includes providing a device including a first portion and a second portion coupled to the first portion. An angle between the first portion and the second portion is greater than approximately 45° such that the first portion is configurable to fit a first bone surface and the second portion is configurable to fit a second bone surface. The first portion is coupled to the first bone surface, and the second portion is coupled to the second bone surface.

In another aspect, an osteosynthesis device is provided. The device includes a first portion and a second portion coupled to the first portion. The first portion is positioned substantially along a first plane. The second portion is positioned substantially along a second plane that is different from the first plane.

In yet another aspect, an osteosynthesis device is provided. The device includes a first portion and a second portion coupled to the first portion. The first portion is configurable to fit a first bone surface. An angle between the first portion and the second portion is greater than approximately 45° and less than approximately 45° such that the second portion is configurable to fit a second bone surface.

### BRIEF DESCRIPTION OF THE DRAWINGS

FIG. 1 is a first perspective view of an exemplary osteosynthesis device according to an embodiment of the present invention;

FIG. 2 is a second perspective view of the osteosynthesis device shown in FIG. 1; and

FIG. 3 is a flow chart illustrating an exemplary method for osteosynthesis using the device shown in FIG. 1.

### DETAILED DESCRIPTION OF THE INVENTION

The methods and apparatus described herein relate to osteosynthesis devices. The osteosynthesis device described herein includes a first portion and a second portion coupled to the first portion. The first portion is positioned substantially along a first plane and is configurable to fit a first bone surface. The second portion is positioned substantially along a second plane that is different from the first plane and is configurable to fit a second bone surface.

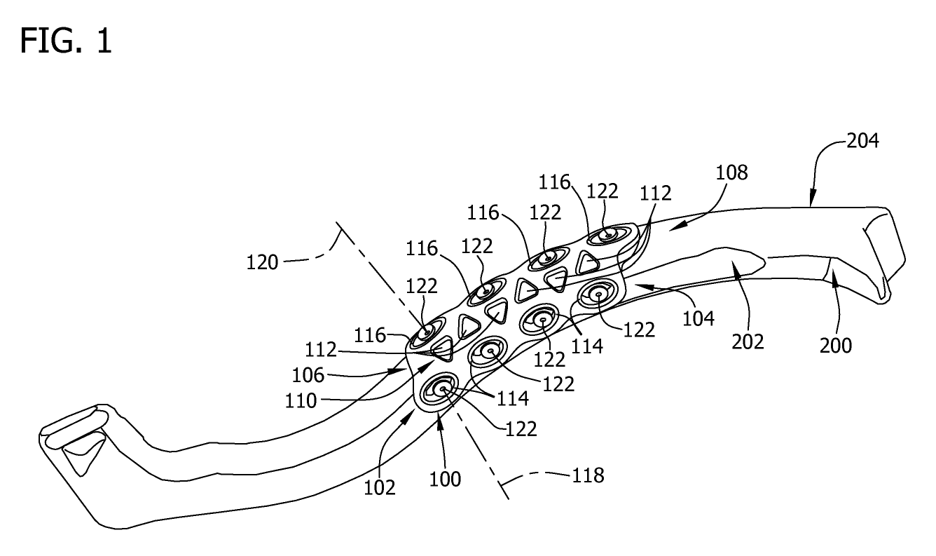

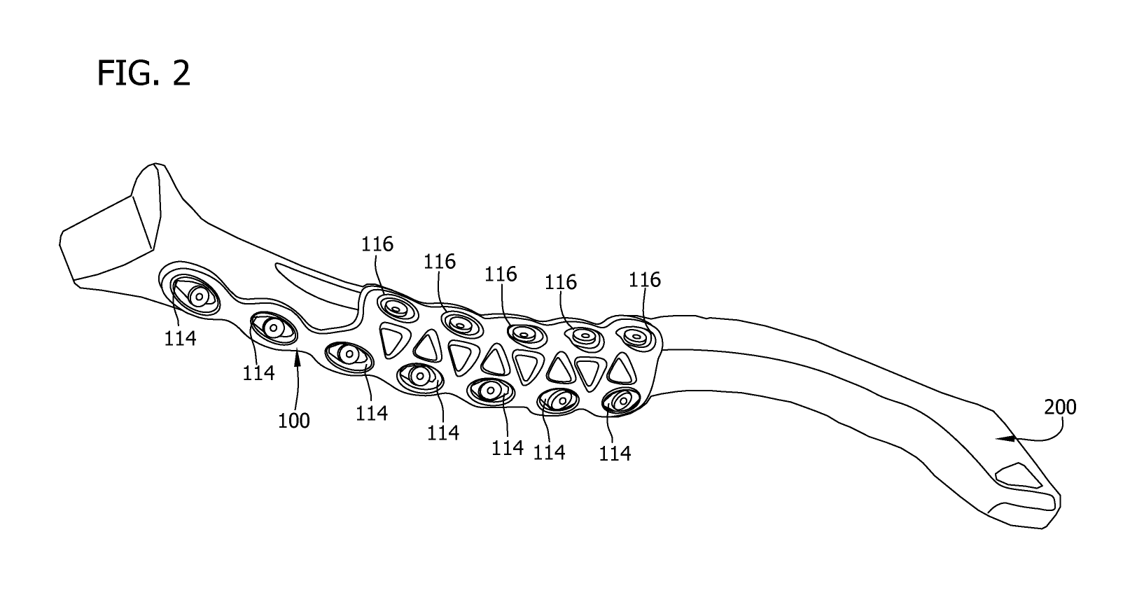

FIG. 1 is a first perspective view of an exemplary osteosynthesis device 100, and FIG. 2 is a second perspective view of device 100. In the exemplary embodiment, device 100 is configured to provide fixation for acute fractures, malunions, and non-unions of a bone 200.

In the exemplary embodiment, device 100 is a biplanar fracture fixation device that includes a first portion 102 positioned substantially along a first plane 104 and a second portion 106 positioned substantially along a second plane 108 that is different from first plane 104. Notably, first portion 102 is not limited to being at any particular angle with respect to second portion 106, but rather may vary depending on anatomic location. Particularly, in the exemplary embodiment, the angle between portions 102 and 106 is between approximately 45° and approximately 135°. More particularly, in the exemplary embodiment, the angle between portions 102 and 106 is between about approximately 60° and approximately 120°. Even more particularly, in the exemplary embodiment, the angle between portions 102 and 106 is between about approximately 75° and approximately 105°. Even more particularly, in the exemplary embodiment, first portion 102 is substantially perpendicular to second portion 106.

In the exemplary embodiment, device 100 is anatomically precontoured to fit bone 200 and, more specifically, to fit a surface of bone 200. More specifically, in the exemplary embodiment, first portion 102 and/or second portion 106 are anatomically contoured to fit at least one of a first bone surface 202 and a second bone surface 204. Notably, device 100 is not limited for use with any particular bone, surface of bone, or bone fracture, but rather is intended for use with a radius, ulna, clavicle, humerus, and/or fibula, as well as other bones.

In the exemplary embodiment, device 100 is fabricated from a material that is suitably malleable to configure, or physically manipulate, device 100 to fit first bone surface 202 and/or second bone surface 204. Moreover, in the exemplary embodiment, device 100 is fabricated from a material that is suitably robust to provide support to bone 200. In the exemplary embodiment, device 100 is fabricated from titanium and/or stainless steel. Further, in the exemplary embodiment, device 100 has a suitable thickness between approximately 2.0 millimeters (mm) and approximately 5.0 mm. Notably, device 100 is not limited to any particular thickness, but rather may vary depending upon anatomic location. For example, a first device 100 configured for use with a humerus has a larger thickness than a second device 100 configured for use with a radius, ulna, and/or fibula.

In the exemplary embodiment, device 100 includes a truss 110 that is positioned between first and second portions 102 and 106. In the exemplary embodiment, truss 110 includes at least one triangular unit that is configured to provide structural support to device 100 and/or bone 200. More specifically, in the exemplary embodiment, truss 110 is configured such that first portion 102 provides structural support to second portion 106, and second portion 106 provides structural support to first portion 102.

In the exemplary embodiment, truss 110 includes a plurality of openings 112. In the exemplary embodiment, openings 112 facilitate decreasing a mass of device 100. In the exemplary embodiment, at least one opening 112 is triangular in shape. Notably, openings 112 are not limited to any particular shape, but rather may vary to enable device 100 to function as described herein.

In the exemplary embodiment, first portion 102 includes a first plurality of openings 114 extending therethrough, and second portion 106 includes a second plurality of openings 116 extending therethrough. Moreover, in the exemplary embodiment, openings 114 define a first plurality of centerlines 118 extending therethrough, and openings 116 define a second plurality of centerlines 120 extending therethrough. In the exemplary embodiment, first plurality of centerlines 118 are substantially perpendicular to first portion 102 and, thus, substantially perpendicular to first plane 104. Moreover, in the exemplary embodiment, second plurality of centerlines 120 are substantially perpendicular to second portion 106 and, thus, substantially perpendicular to second plane 108.

In the exemplary embodiment, at least one of first plurality of centerlines 118 does not intersect with any of second plurality of centerlines 120. More specifically, in the exemplary embodiment, each of first plurality of centerlines 118 does not intersect with any of second plurality of centerlines 120. In the exemplary embodiment, first plurality of openings 114 include a first quantity of openings, and second plurality of openings 116 include a second quantity of openings that is different form the first quantity of openings. Notably, openings 114 and 116 are not limited to any particular quantity, but rather may vary to enable device 100 to function as described herein.

In the exemplary embodiment, at least one opening 114 and/or opening 116 has a circular shape, an oval shape, or an elliptical shape. Moreover, in the exemplary embodiment, at least one opening 114 and/or opening 116 is configured to receive at least one coupling mechanism 122 to facilitate coupling device 100 to bone 200. Notably, the biplanar configuration of device 100 enables using a smaller caliber coupling mechanism 122 than known osteosynthesis devices. In the exemplary embodiment, coupling mechanism 122 may or may not lock to device 100. More specifically, in the exemplary embodiment, coupling mechanism 122 includes a pin, a nail, a screw, and/or a locking screw. Notably, each of first plurality of openings 114 and/or second plurality of openings 116 is not limited to receive any particular coupling mechanism, but rather may be configured to receive a coupling mechanism of a variety of shapes and/or sizes.

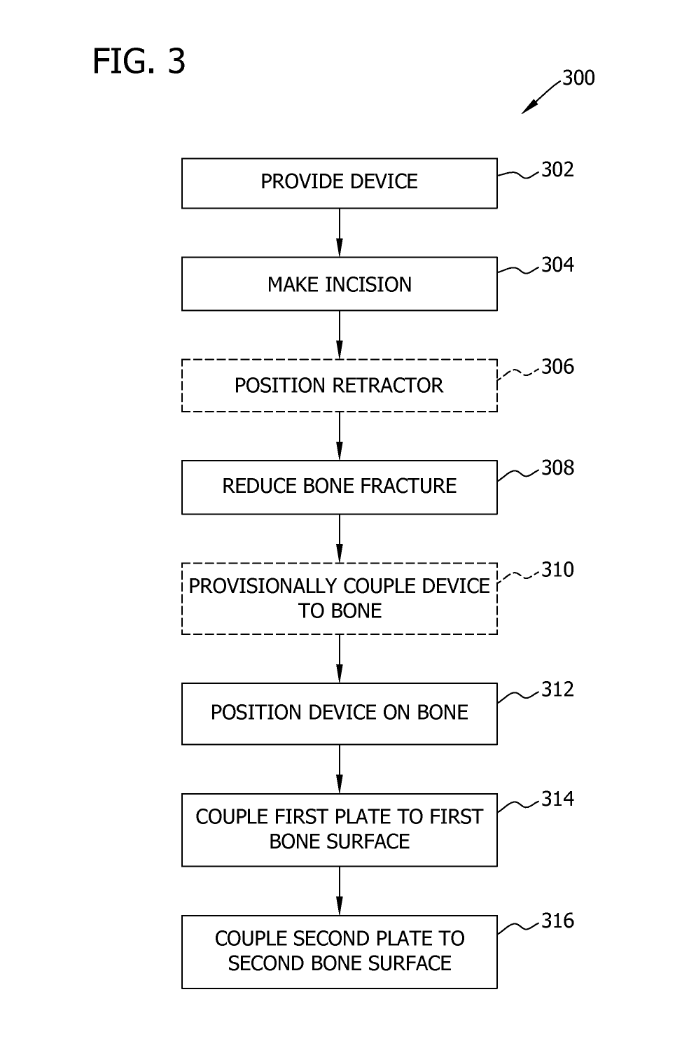

FIG. 3 is a flow chart illustrating an exemplary method 300 for osteosynthesis using device 100. In the exemplary embodiment, a suitable device 100 is provided 302 and/or selected based on type of bone, size of bone, and/or location of a bone fracture.

During operation, an incision is made 304 over the bone fracture. More specifically, in the exemplary embodiment, an incision is made 304 to enable positioning and/or coupling device 100 to bone 200. Particularly, the incision is less than approximately 12.0 centimeters (cm). More particularly, the incision is less than approximately 9.0 cm. Even more particularly, the incision is less than approximately 6.0 cm. Alternatively, the incision may be longer than approximately 12.0 cm. Notably, the biplanar configuration of device 100 enables device 100 to have a shorter length than known osteosynthesis devices that are planar. For example, in the exemplary embodiment, the configuration of first and second pluralities of openings 114 and 116 on first and second portions 102 and 106 enables coupling device 100 to bone 200 within a shorter length for a given number of coupling mechanisms 122 than known osteosynthesis devices. As such, a shorter incision is required to position and/or couple device 100 to bone 200 than known osteosynthesis devices. A shorter incision includes several benefits including less damage and/or irritation to surrounding tissue, fewer stitches to close the incision, and potentially avoiding neurovascular tissue. Moreover, in the exemplary embodiment, first and second portions 102 and 106 provide a robust biplanar support to bone 200.

In the exemplary embodiment, a retractor (not shown) is positioned 306 such that neurovascular structures are protected from coupling mechanism 122, and the bone fracture is reduced 308. In the exemplary embodiment, at least one clamp (not shown) is used to provisionally couple 310 device 100 to first bone surface 202 and/or second bone surface 204, and device 100 is positioned 312 on first bone surface 202 and/or second bone surface 204.

In the exemplary embodiment, at least one coupling mechanism 122 is inserted through first plurality of openings 114 to securely couple 314 first portion 102 to first bone surface 202, and at least one coupling mechanism 122 is inserted through second plurality of openings 116 to securely couple 316 second portion 106 to second bone surface 204. For example, in the exemplary embodiment, coupling mechanisms 122 are inserted through first and second pluralities of openings 114 and 116 in an alternating fashion. Alternatively, at least one coupling mechanism 122 may be inserted through at least one of first plurality of opening 114 and then at least one coupling mechanism 122 may be inserted through at least one of second plurality of openings 116. Notably, the insertion of coupling mechanisms 122 are not limited to any particular sequence, but rather may be inserted in any suitable sequence of inserting coupling mechanisms 122.

In the exemplary embodiment, at least one coupling mechanism 122 inserted through at least one of first plurality of openings 114 is directed towards, converges upon, interfaces with, and/or locks with at least one coupling mechanism 122 inserted through at least one of second plurality of openings 116. In the exemplary embodiment, at least one of first plurality of centerlines 118 deviates from each of second plurality of centerlines 120 to prevent coupling mechanisms 122 from intersecting within bone 200 upon insertion of coupling mechanisms 122 through first plurality of openings 114 and/or second plurality of openings 116.

Once device 100 is securely coupled to bone 200, in the exemplary embodiment, the bone clamps are removed, the musculature is re-approximated, and the skin is closed with stitches.

The methods and systems described herein relate to osteosynthesis devices. The osteosynthesis device described herein includes a first portion and a second portion coupled to the first portion. The first portion is positioned substantially along a first plane and is configurable to fit a first bone surface. The second portion is positioned substantially along a second plane that is different from the first plane and is configurable to fit a second bone surface. The exemplary embodiments described herein provide resistance to a plurality of forces in a plurality of planes. Additionally, the exemplary embodiments described herein provide a stiffer fracture fixation, a fixation of comminuted fragments, a smaller caliber of screw, a shorter incision, and/or a shorter portion.

Exemplary embodiments of osteosynthesis devices are described above in detail. The methods and systems are not limited to the specific embodiments described herein, but rather, operations of the methods and components of the systems may be utilized independently and separately from other operations and/or components described herein. For example, the methods and apparatus described herein may have other industrial and/or consumer applications and are not limited to practice with osteosynthesis as described herein. Rather, one or more embodiments may be implemented and utilized in connection with other industries.

As used herein, an element or step recited in the singular and proceeded with the word “a” or “an” should be understood as not excluding plural said elements or steps, unless such exclusion is explicitly stated. Further, references to “one embodiment” are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for osteosynthesis, said method comprising:
providing a device including a first portion and a second portion coupled to the first portion, wherein an angle between the first portion and the second portion is greater than approximately 45° such that the first portion is configurable to fit a first bone surface and the second portion is configurable to fit a second bone surface;
coupling the first portion to the first bone surface; and
coupling the second portion to the second bone surface.

2. A method for osteosynthesis in accordance with claim 1 further comprising making an incision of less than approximately 12.0 cm.

3. A method for osteosynthesis in accordance with claim 1 further comprising provisionally coupling the device to at least one of the first and second bone surfaces using at least one clamp.

4. A method for osteosynthesis in accordance with claim 1, wherein providing a device further comprises including a first plurality of openings extending through the first portion and including a second plurality of openings extending through the second portion, wherein coupling the first portion further comprises inserting a first plurality of coupling mechanisms through the first plurality of openings, and wherein coupling the second portion further comprises inserting a second plurality of coupling mechanisms through the second plurality of openings.

5. A method for osteosynthesis in accordance with claim 4, wherein at least one of the first plurality of coupling mechanisms do not intersect with any of the second plurality of coupling mechanisms.

6. A method for osteosynthesis in accordance with claim 4, wherein each of the first plurality of coupling mechanisms do not intersect with any of the second plurality of coupling mechanisms.

7. An osteosynthesis device comprising:
a first portion positioned substantially along a first plane; and
a second portion coupled to the first portion, the second portion positioned substantially along a second plane that is different from the first plane.

8. An osteosynthesis device in accordance with claim 7 further comprising a truss that is positioned between the first and second portions, wherein the truss includes a plurality of truss openings.

9. An osteosynthesis device in accordance with claim 7, wherein the first portion includes a first plurality of openings extending therethrough, and the second portion includes a second plurality of openings extending therethrough.

10. An osteosynthesis device in accordance with claim 9, wherein the first plurality of openings define a first plurality of centerlines extending therethrough, and the second plurality of openings define a second plurality of centerlines extending therethrough, wherein at least one of the first plurality of centerlines do not intersect with any of the second plurality of centerlines.

11. An osteosynthesis device in accordance with claim 9, wherein the first plurality of openings define a first plurality of centerlines extending therethrough, and the second plurality of openings define a second plurality of centerlines extending therethrough, wherein each of the first plurality of centerlines do not intersect with any of the second plurality of centerlines.

12. An osteosynthesis device in accordance with claim 9, wherein the first plurality of openings include a first quantity of openings and the second plurality of openings include a second quantity of openings that is different form the first quantity of openings.

13. An osteosynthesis device in accordance with claim 7, wherein an angle between the first plane and the second plane is between approximately 45° and approximately 135°.

14. An osteosynthesis device in accordance with claim 7, wherein the first plane is substantially perpendicular to the second plane.

15. An osteosynthesis device comprising:
a first portion configurable to fit a first bone surface; and
a second portion coupled to the first portion, wherein an angle between the first portion and the second portion is greater than approximately 45° and less than approximately 45° such that the second portion is configurable to fit a second bone surface.

16. An osteosynthesis device in accordance with claim 15 further comprising a truss that is positioned between the first and second portions, wherein the truss includes a plurality of truss openings.

17. An osteosynthesis device in accordance with claim 15, wherein the first portion includes a first plurality of openings extending therethrough, and the second portion includes a second plurality of openings extending therethrough.

18. An osteosynthesis device in accordance with claim 17, wherein the first plurality of openings define a first plurality of centerlines extending therethrough, and the second plurality of openings define a second plurality of centerlines extending therethrough, wherein at least one of the first plurality of centerlines do not intersect with any of the second plurality of centerlines.

19. An osteosynthesis device in accordance with claim 17, wherein the first plurality of openings define a first plurality of centerlines extending therethrough, and the second plurality of openings define a second plurality of centerlines extending therethrough, wherein each of the first plurality of centerlines do not intersect with any of the second plurality of centerlines.

20. An osteosynthesis device in accordance with claim 17, wherein the first plurality of openings include a first quantity of openings and the second plurality of openings include a second quantity of openings that is different form the first quantity of openings.
